(19) United States
(12) Patent Application Publication
(10) Pub. No.: US 2025/0195550 A1
(43) Pub. Date: Jun. 19, 2025

(54) A NOVEL NUTRACEUTICAL/THERAPEUTICAL COMPOSITION OF CINNAMIC ACID DERIVATIVES WITH SUCROSE ISOMERS

(71) Applicant: FERTIS INDIA PVT. LTD., Hyderabad, Telangana (IN)

(72) Inventors: Rahul Raju KANUMURU, Hyderabad, Telangana (IN); Shaji George KOCHUMALAYIL, Hyderabad, Telangana (IN); Ravikumar SURANENI, Hyderabad, Telangana (IN); Murali Mohanarao VANA, Hyderabad, Telangana (IN); Prasannakumar Lakshmi GARUDADHIRI, Hyderabad, Telangana (IN)

(73) Assignee: FERTIS INDIA PVT. LTD., Hyderabad, Telangana (IN)

(21) Appl. No.: 18/851,717

(22) PCT Filed: Mar. 30, 2023
(86) PCT No.: PCT/IN2023/050316

§ 371 Date: Sep. 27, 2024

(30) Foreign Application Priority Data
Mar. 31, 2022 (IN) 202241019382

(51) Int. Cl. A61K 31/7016; A61K 9/19; A61K 31/165; A61K 31/192; A61K 31/216; A61K 31/4045; A61K 31/7004

(52) U.S. Cl. CPC A61K 31/7016; A61K 9/19; A61K 31/165; A61K 31/192; A61K 31/216; A61K 31/4045; A61K 31/7004

(57) ABSTRACT

The invention discloses Nutraceutical/Therapeutical compositions comprising Cinnamic acid derivatives with sucrose isomers useful as nootropic performance enhancers and also for the pain management. The invention further discloses process for the preparation of the said compositions and methods of use thereof.

### TECHNICAL FIELD

The invention relates to Nutraceutical/Therapeutical compositions comprising Cinnamic acid derivatives with sucrose isomers useful as nootropic performance enhancers and also for the pain management. The invention further relates to process for the preparation of the said compositions and methods of use thereof.

### BACKGROUND AND PRIOR ART

HydroxyCinnamic acids are phenolic acid compounds which are present in many plants either in free state or as derivatives such as esters or amides and also known to possess potent antioxidant, antitumor, antidepressant, cytoprotective neuroinflammation in neurodegenerative diseases, anti-inflammatory and antiarthritic properties. They also prevent insulin resistance, cardiovascular disease, renal dysfunctions, diabetes, dyslipidemia and improves liver functions. Furthermore, esters of hydroxy Cinnamic acids have potential therapeutic applications in experimental diabetes and hyperlipidemia, lowering lipid profile, improving brain functions and pain management.

Naturally occurring esters of hydroxy Cinnamic acids are known to reduce the anxiety and stress by regulating GABA receptors. Cinnamic acids esters are known to increase the firing of neurons which leads to the improvement of brain function and memory. WO2020176432A1 discloses a method for inhibiting the progression of glycine encephalopathy comprising, administering to a patient in need thereof an therapeutically effective amount of a pharmaceutical composition comprising Cinnamic acid.

CN107624068A discloses method for treating hypertension using a combination comprising Cinnamic acid and zinc to individual in need thereof.

Isomers of sugars are also involved in various metabolic and signaling pathways, including those that contribute to antioxidant properties, scavengers of hydroxyl radicals, their effectiveness against inflammation, as neuroprotectant. These sugars also have many physiological and cardiometabolic benefits, ranging from weight loss to improving glycemic control and reducing insulin resistance. The health benefits of rare sugars in humans have begun to accrue for a number of rare sugars, including allulose, tagatose, isomaltulose, arabinose, and trehalose.

Currently, naturally occurring sucrose isomers, trehalose, trehalulose, turanose, leucrose, and isomaltulose are considered to be possible alternatives to sucrose due to their potential cognitive benefits like positive effects on mood, improved episodic and working memory, improved attention speed. In addition, sucrose isomers also contribute in fat oxidation in energy metabolism; improved physical performance; protection against hypoglycemia during exercise.

Evidence of the facilitation of episodic memory suggests a specific enhancing effect of sucrose isomers intake on cognitive domains associated with the function of the hippocampus. In support of this, facilitative effects have also been reported for additional hippocampal-dependent cognitive functions like recognition memory; visuospatial memory and visuospatial functioning.

The antioxidant activity of the rare sugar D-allose by eliminating the hydroxyl radicals has recently been reported and the effect is close to the extent D glucose scavenge the hydroxyl radicals. Cancer and tumor inhibition is considered to be the most important property of D-allose. D-allose has been reported to be effective against various human cancers, such as ovarian, cervical and skin, hepatocellular and prostate cancers. Leukemia, head and neck cancer, pancreas, and lung cancer are also inhibited by D allose.

The anti-oxidant activity of D-allulose persists over a long period of storage, and is highly soluble compared to D fructose or D glucose. D-allulose showed hypoglycemic properties and therapeutic effects on type 2 diabetes. It also has anti-obesity and antihyperlipidemic effects. Furthermore, D-allulose can be used against inflammation and atherosclerotic diseases, as a neuroprotectant.

D-tagatose has antioxidant and cryoprotectant properties in addition to its ability to assist in body weight reduction. D-tagatose has also therapeutic potential in type 2 diabetes management, since it improves glycemic control.

Although the applications of rare sugars in human nutrition and medicine have been widely studied, their potential use for sustainable food production, and application in agriculture is scantly reported.

Despite the potential benefit of these natural products there exist hardly any literature available in the prior art or a product available in the market for human application which offers the synergistic properties of Cinnamic acid and isomers of sugars in general and sucrose in particular.

Accordingly, a provision of a composition comprising the combination of Cinnamic acid and its derivatives with sugar isomers have become an objective of the present invention owing to their structural diversity, together with distinctive and remarkable pharmacological and pharmacodynamic actions, such as antidepression, anticancer, antioxidant, anti-inflammatory and antiviral activities.

Therefore, the inventors felt that there is a need in the art to develop novel formulation(s) which will help in improving focus and learning, reduce anxiety, improves physical and mental performance, effectively manage inflammatory disease and pain and also act as scavengers of reactive oxygen species thereby offering cell protection.

### SUMMARY OF THE INVENTION

In line with the above objective, the present invention provides unique dual role of novel Nutraceutical/Therapeutical compositions/formulations comprising Cinnamic acids or its derivatives with sucrose isomers useful as nootropic performance enhancers.

Accordingly, the invention provides novel compositions/formulations comprising Cinnamic acid derivatives with Sucrose isomers having nutritive/therapeutic value, wherein, the Cinnamic acid or its derivatives present in an amount of 2%-90% w/w and sucrose isomers present in an amount of 10-90% w/w.

In another aspect, the composition also contains suitable excipients, which can be selected from the group consisting of binders, diluents, lubricants, stabilizers, solubilizers, sustained release polymers, suitable coloring agents, taste enhancers, etc., to obtain desired formulation properties.

In a further aspect, the Nutraceutical/Therapeutical composition can be formulated using suitable excipients into either oral solid, liquid or suspension formulations using suitable techniques.

In yet another aspect, the invention provides methods of improving focus and learning, reducing anxiety, improving physical and mental performance, effectively managing inflammatory disease & pain and for scavenging the reactive oxygen species thereby offering cell protection in a subject, which method comprises treating the said subject with a composition comprising Cinnamic Acids or its derivatives with Sucrose Isomers in therapeutically effective amounts.

### DETAILED DESCRIPTION OF THE INVENTION

The invention will now be described in detail in connection with certain preferred and optional embodiments, so that various aspects thereof may be more fully understood and appreciated.

Accordingly, the present invention provides novel Nutraceutical/Therapeutical compositions/formulations comprising Cinnamic acids or its derivatives with Sucrose isomers useful as nootropic performance enhancers and also for the pain management.

The term “Nutraceutical” refers to a hybrid of ‘nutrition’ and ‘pharmaceutical’. Nutraceuticals, in broad, are ‘functional food’ or part of “food playing a significant role in modifying and maintaining normal physiological function that maintains health of human beings”.

Accordingly, the invention provides novel nutraceutical compositions/formulations comprising Cinnamic acid derivatives with sugar isomers having nutritive/therapeutic value, wherein, the Cinnamic acid or its derivatives present in an amount of 2%-90% w/w and Sucrose isomers present in an amount of 10-90% w/w and nutraceutical/therapeutic excipient in an amount of 5-90%.

In another aspect, the Nutraceutical/Therapeutical compositions of the present invention can also be useful for pain management.

In one of the embodiments, the Cinnamic acid derivatives are selected from Hydroxy Cinnamic acids, its esters and amides.

In an embodiment, the Hydroxy Cinnamic acid derivatives (CA) are selected from Hydroxy Cinnamic acid, Ferulic acid, Caffeic acid, Sinapic acid, Chlorogenic Acid, Rosmarinic acid, Syringic acid, p-Coumaric acid etc.

In another embodiment, the derivatives of Cinnamic acid esters are selected from alkyl/aralkyl alcohols esterified with appropriate Cinnamic acids having general formula CA-CO—OR where CA=Cinnamic Acid and R=C1-C22 alkyl chain; Ph-CH₂CH₂— and CA-CO—O(CH)nO-CO-CA where n=1-10. Esters of Cinnamic acids also includes branched alkyl groups such as Isopropyl, Iso-butyl cinnamate, Isopentyl, Phenylethyl etc.

In yet another embodiment, the derivatives of Cinnamic acid amides are selected from amides having general formula CA-CO—NHR where CA-CO—=Cinnamic Acid and NHR=Tyramine, Phenethylamine, Serotonin or Dopamine

In another embodiment, the isomers of sugars are selected from monosaccharides or disaccharides. Isomers of monosaccharides sugars are selected from D Allose, D Allulose and D Tagatose.

The isomers of disaccharides sugars are selected from Trehalulose, Turanose, Leucrose and Isomaltulose.

In another embodiment, the composition also contains suitable nutraceutical/therapeutic excipients, which can be selected from the group consisting of binders, diluents, lubricants, stabilizers, solubilizers, sustained release polymers, suitable coloring agents, taste enhancers, etc.

In a further embodiment, the composition can be formulated using suitable excipients into either oral solid, liquid or suspension formulations.

In another embodiment, the nutraceutical/therapeutic composition of the present may be prepared by a process known in the art comprising adding suitable excipients to the active ingredients Cinnamic acid derivatives along with sucrose isomers in suitable proportions.

Accordingly, the process for preparation of the present composition comprises;
i. Adding cinnamic acid derivatives in an amount of 2%-90% w/w to the aqueous solution of the sugar isomers in an amount of 10%-90% w/w;
ii. Adding suitable nutraceutical excipients in an amount of 5-90% w/w to the solution of step (i); and
iii. Agitating the solution of step (ii) to avoid heat-induced inversion of sucrose followed by drying to obtain the desired composition.

In yet another embodiment, the invention provides method of enhancing the brain performance, reducing anxiety, improving the learning and focus without anxiety in a subject which method comprises treating the said subject with a composition comprising Cinnamic Acids or its derivatives with Sucrose Isomers in nutraceutically effective amounts.

In yet another embodiment, the invention provides method of treating inflammation in a subject which method comprises treating the said subject with a composition comprising Cinnamic Acids or its derivatives with Sucrose Isomers in therapeutically effective amounts, wherein the inflammation is selected from the inflammation related to the COX 2.

The term “nutraceutically effective amount” means an amount that renders desired nutritional and therapeutic benefit of the or outcome in alleviating or reducing or controlling the progression of the disease.

The following examples are presented to further explain the invention with experimental conditions, which are purely illustrative and are not intended to limit the scope of the invention.

### EXAMPLES

### Illustrative Formulations:

The formulations listed below are set forth for illustrative purposes only, and should not be used to limit the proper construction of the claims in any manner whatsoever. In the formulations nutraceutical excipient percentage varies from 5-90%

### Illustrative Formulations are Set Forth Below:

### Example 1: Formulation of Cinnamic Acid Derivatives with Mono and Disaccharides

| | | | | Percentage |
|---|---|---|---|---|
| | | Percentage | Cinnamic Acid | of Cinnamic |
| S. NO | Sugar | of Sugar | derivative | acid |
| 1 | D-allose | 10-90 | Cinnamic acid | 2-90 |
| | | | Ferulic acid | |
| | | | Caffeic acid | |
| | | | Sinapic acid | |
| | | | Chlorogenic Acid | |
| | | | Rosmarinic acid | |
| | | | Syringic acid | |
| | | | p-Coumaric acid | |
| 2 | D-Allulose | 10-90 | Cinnamic acid | 2-90 |
| | | | Ferulic acid | |
| | | | Caffeic acid | |
| | | | Sinapic acid | |
| | | | Chlorogenic Acid | |
| | | | Rosmarinic acid | |
| | | | Syringic acid | |
| | | | p-Coumaric acid | |
| 3 | D-Tagatose | 10-90 | Cinnamic acid | |
| | | | Ferulic acid | |
| | | | Caffeic acid | 2-90 |
| | | | Sinapic acid | |
| | | | Chlorogenic Acid | |
| | | | Rosmarinic acid | |
| | | | Syringic acid | |
| | | | p-Coumaric acid | |
| 4 | Trehalulose | 10-90 | Cinnamic acid | 2-90 |
| | | | Ferulic acid | |
| | | | Caffeic acid | |
| | | | Sinapic acid | |
| | | | Chlorogenic Acid | |
| | | | Rosmarinic acid | |
| | | | Syringic acid | |
| | | | p-Coumaric acid | |
| 5 | Turanose | 10-90 | Cinnamic acid | 2-90 |
| | | | Ferulic acid | |
| | | | Caffeic acid | |
| | | | Sinapic acid | |
| | | | Chlorogenic Acid | |
| | | | Rosmarinic acid | |
| | | | Syringic acid | |
| | | | p-Coumaric acid | |
| 6 | Leucrose | 10-90 | Cinnamic acid | 2-90 |
| | | | Ferulic acid | |
| | | | Caffeic acid | |
| | | | Sinapic acid | |
| | | | Chlorogenic Acid | |
| | | | Rosmarinic acid | |
| | | | Syringic acid | |
| | | | p-Coumaric acid | |
| 7 | Isomaltulose | 10-90 | Cinnamic acid | 2-90 |
| | | | Ferulic acid | |
| | | | Caffeic acid | |
| | | | Sinapic acid | |
| | | | Chlorogenic Acid | |
| | | | Rosmarinic acid | |
| | | | Syringic acid | |
| | | | p-Coumaric acid | |

### Example 2: Formulation of Cinnamic Acid Ester Derivatives with Mono and Disaccharides

| | | Percent- | | Percentage |
|---|---|---|---|---|
| | | age of | Cinnamic Acid ester | of Cinnamic |
| S. NO | Sugar | Sugar | derivative | acid |
| 1 | D-allose | 10-90 | n-Hexadecyl Cinnamte | 2-90 |
| | | | n-Octadecyl cinnamte | |
| | | | n-Eicosyl cinnamate | |
| | | | n-Docosyl cinnamate | |
| | | | n-Hexadecyl ferulate | |
| | | | n-Octadecyl ferulate | |
| | | | n-Eicosyl ferulate | 2-90 |
| | | | n-Docosyl ferulate | |
| | | | n-Hexadecyl caffeate | |
| | | | n-Octadecyl caffeate | |
| | | | n-Eicosyl caffeate | |
| | | | n-Docosyl caffeate | |
| | | | n-Hexadecyl coumarate | |
| | | | n-Octadecyl coumarate | |
| | | | n-Eicosyl coumarate | |
| | | | n-Docosyl coumarate | |
| | | | Caffeic acid phenethyl | |
| | | | ester | |
| 2 | D-Allulose | 10-90 | n-Hexadecyl cinnamte | 2-90 |
| | | | n-Octadecyl cinnamte | |
| | | | n-Eicosyl cinnamate | |
| | | | n-Docosyl cinnamate | |
| | | | n-Hexadecyl ferulate | |
| | | | n-Octadecyl ferulate | |
| | | | n-Eicosyl ferulate | |
| | | | n-Docosyl ferulate | |
| | | | n-Hexadecyl caffeate | |
| | | | n-Octadecyl caffeate | |
| | | | n-Eicosyl caffeate | |
| | | | n-Docosyl caffeate | |
| | | | n-Hexadecyl coumarate | |
| | | | n-Octadecyl coumarate | |
| | | | n-Eicosyl coumarate | |
| | | | n-Docosyl coumarate | |
| | | | Caffeic acid phenethyl | |
| | | | ester | |
| 3 | D-Tagatose | 10-90 | n-Hexadecyl cinnamte | 2-90 |
| | | | n-Octadecyl cinnamte | |
| | | | n-Eicosyl cinnamate | |
| | | | n-Docosyl cinnamate | |
| | | | n-Hexadecyl ferulate | |
| | | | n-Octadecyl ferulate | |
| | | | n-Eicosyl ferulate | |
| | | | n-Docosyl ferulate | |
| | | | n-Hexadecyl caffeate | |
| | | | n-Octadecyl caffeate | |
| | | | n-Eicosyl caffeate | |
| | | | n-Docosyl caffeate | |
| | | | n-Hexadecyl coumarate | |
| | | | n-Octadecyl coumarate | |
| | | | n-Eicosyl coumarate | |
| | | | Caffeic acid phenethyl | |
| | | | ester | |
| | | | n-Docosyl coumarate | |
| 4 | Trehalulose | 10-90 | n-Hexadecyl cinnamte | 2-90 |
| | | | n-Octadecyl cinnamte | |
| | | | n-Eicosyl cinnamate | |
| | | | n-Docosyl cinnamate | |
| | | | n-Hexadecyl ferulate | |
| | | | n-Octadecyl ferulate | |
| | | | n-Eicosyl ferulate | |
| | | | n-Docosyl ferulate | |
| | | | n-Hexadecyl caffeate | |
| | | | n-Octadecyl caffeate | |
| | | | n-Eicosyl caffeate | |
| | | | n-Docosyl caffeate | |
| | | | n-Hexadecyl coumarate | |
| | | | n-Octadecyl coumarate | |
| | | | n-Eicosyl coumarate | |
| | | | Caffeic acid phenethyl | |
| | | | ester | |
| | | | n-Docosyl coumarate | |
| 5 | Turanose | 10-90 | n-Hexadecyl cinnamte | 2-90 |
| | | | n-Octadecyl cinnamte | |
| | | | n-Eicosyl cinnamate | |
| | | | n-Docosyl cinnamate | |
| | | | n-Hexadecyl ferulate | |
| | | | n-Octadecyl ferulate | |
| | | | n-Eicosyl ferulate | |
| | | | n-Docosyl ferulate | |
| | | | n-Hexadecyl caffeate | |
| | | | n-Octadecyl caffeate | |
| | | | n-Eicosyl caffeate | |
| | | | n-Docosyl caffeate | |
| | | | n-Hexadecyl coumarate | |
| | | | n-Octadecyl coumarate | |
| | | | n-Eicosyl coumarate | |
| | | | Caffeic acid phenethyl | |
| | | | ester | |
| | | | n-Docosyl coumarate | |
| 6 | Leucrose | 10-90 | n-Hexadecyl cinnamte | 2-90 |
| | | | n-Octadecyl cinnamte | |
| | | | n-Eicosyl cinnamate | |
| | | | n-Docosyl cinnamate | |
| | | | n-Hexadecyl ferulate | |
| | | | n-Octadecyl ferulate | |
| | | | n-Eicosyl ferulate | |
| | | | n-Docosyl ferulate | |
| | | | n-Hexadecyl caffeate | |
| | | | n-Octadecyl caffeate | |
| | | | n-Eicosyl caffeate | |
| | | | n-Docosyl caffeate | |
| | | | n-Hexadecyl coumarate | |
| | | | n-Octadecyl coumarate | |
| | | | n-Eicosyl coumarate | |
| | | | Caffeic acid phenethyl | |
| | | | ester | |
| | | | n-Docosyl coumarate | |
| 7 | Isomaltulose | 10-90 | n-Hexadecyl cinnamte | 2-90 |
| | | | n-Octadecyl cinnamte | |
| | | | n-Eicosyl cinnamate | |
| | | | n-Docosyl cinnamate | |
| | | | n-Hexadecyl ferulate | |
| | | | n-Octadecyl ferulate | |
| | | | n-Eicosyl ferulate | |
| | | | n-Docosyl ferulate | |
| | | | n-Hexadecyl caffeate | |
| | | | n-Octadecyl caffeate | |
| | | | n-Eicosyl caffeate | |
| | | | n-Docosyl caffeate | |
| | | | n-Hexadecyl coumarate | |
| | | | n-Octadecyl coumarate | |
| | | | n-Eicosyl coumarate | |
| | | | Caffeic acid phenethyl | |
| | | | ester | |
| | | | n-Docosyl coumarate | |

### Example 3: Formulation of Cinnamic Acid Amide Derivatives with Mono and Disaccharides

| | | | | Percentage |
|---|---|---|---|---|
| | | Percentage | Cinnamic Acid | of Cinnamic |
| S. NO | Sugar | of Sugar | amide derivative | acid |
| 10 | D-allose | 10-90 | Cinnamyl tyramine | 2-90 |
| | | | Feruloyl tyramine | |
| | | | Coumaroyl tyramine | |
| | | | Caffeoyl tyramine | |
| | | | Cinnamyl serotonin | |
| | | | Feruloyl serotonin | |
| | | | Coumaroyl serotonin | |
| | | | Caffeoyl serotonin | |
| | | | Caffeic acid | |
| | | | phenethyl amide | |
| | | | Cinnamyl dopamine | |
| | | | Feruloyl dopamine | |
| | | | Coumaroyl | |
| | | | dopamine | |
| | | | Caffeoyl dopamine | |
| 2 | D-Allulose | 10-90 | Cinnamyl tyramine | 2-90 |
| | | | Feruloyl tyramine | |
| | | | Coumaroyl tyramine | |
| | | | Caffeoyl tyramine | |
| | | | Cinnamyl serotonin | |
| | | | Feruloyl serotonin | |
| | | | Coumaroyl serotonin | |
| | | | Caffeoyl serotonin | |
| | | | Caffeic acid phenethyl | |
| | | | amide | |
| | | | Cinnamyl dopamine | |
| | | | Feruloyl dopamine | |
| | | | Coumaroyl dopamine | |
| | | | Caffeoyl dopamine | |
| 3 | D-Tagatose | 10-90 | Cinnamyl tyramine | 2-90 |
| | | | Feruloyl tyramine | |
| | | | Coumaroyl tyramine | |
| | | | Caffeoyl tyramine | |
| | | | Cinnamyl serotonin | |
| | | | Feruloyl serotonin | |
| | | | Coumaroyl serotonin | |
| | | | Caffeoyl serotonin | |
| | | | Caffeic acid phenethyl | |
| | | | amide | |
| | | | Cinnamyl dopamine | |
| | | | Feruloyl dopamine | |
| | | | Coumaroyl dopamine | |
| | | | Caffeoyl dopamine | |
| 4 | Trehalulose | 10-90 | Cinnamyl tyramine | 2-90 |
| | | | Feruloyl tyramine | |
| | | | Coumaroyl tyramine | |
| | | | Caffeoyl tyramine | |
| | | | Cinnamyl serotonin | |
| | | | Feruloyl serotonin | |
| | | | Coumaroyl serotonin | |
| | | | Caffeoyl serotonin | |
| | | | Caffeic acid phenethyl | |
| | | | amide | |
| | | | Cinnamyl dopamine | |
| | | | Feruloyl dopamine | |
| | | | Coumaroyl dopamine | |
| | | | Caffeoyl dopamine | |
| 5 | Turanose | 10-90 | Cinnamyl tyramine | 2-90 |
| | | | Feruloyl tyramine | |
| | | | Coumaroyl tyramine | |
| | | | Caffeoyl tyramine | |
| | | | Cinnamyl serotonin | |
| | | | Feruloyl serotonin | |
| | | | Coumaroyl serotonin | |
| | | | Caffeoyl serotonin | |
| | | | Caffeic acid phenethyl | |
| | | | amide | |
| | | | Cinnamyl dopamine | |
| | | | Feruloyl dopamine | |
| | | | Coumaroyl dopamine | |
| | | | Caffeoyl dopamine | |
| 6 | Leucrose | 10-90 | Cinnamyl tyramine | 2-90 |
| | | | Feruloyl tyramine | |
| | | | Coumaroyl tyramine | |
| | | | Caffeoyl tyramine | |
| | | | Cinnamyl serotonin | |
| | | | Feruloyl serotonin | |
| | | | Coumaroyl serotonin | |
| | | | Caffeoyl serotonin | |
| | | | Caffeic acid phenethyl | |
| | | | amide | |
| | | | Cinnamyl dopamine | |
| | | | Feruloyl dopamine | |
| | | | Coumaroyl dopamine | |
| | | | Caffeoyl dopamine | |
| 7 | Isomaltulose | 10-90% | Cinnamyl tyramine | 2-90% |
| | | | Feruloyl tyramine | |
| | | | Coumaroyl tyramine | |
| | | | Caffeoyl tyramine | |
| | | | Cinnamyl serotonin | |
| | | | Feruloyl serotonin | |
| | | | Coumaroyl serotonin | |
| | | | Caffeoyl serotonin | |
| | | | Caffeic acid phenethyl | |
| | | | amide | |
| | | | Cinnamyl dopamine | |
| | | | Feruloyl dopamine | |
| | | | Coumaroyl dopamine | |
| | | | Caffeoyl dopamine | |
| | | | Cinnamyl tyramine | |

### Example 4: General Procedure for the Preparation of Formulation of Cinnamic Acid Derivatives with Sucrose Isomers

To the aqueous solution of D-Allose, D-Allulose and D-Tagatose, L-Arabinose, Trehalulose, Turanose, Leucrose and Isomaltulose (90-10% w/w) was added, Cinnamic acid/ester/amide (2-90% w/w), followed by the addition of suitable nutraceutical excipients (5-90% w/w). The composition was agitated at room temperature to avoid heat-induced inversion of sucrose for 1.0-2.0 h. The resulting solution/suspension was packed for usage.

### Example 4a

25 g of Trehalulose was added to 100 mL purified water under stirring at room temperature. Slowly added 0.1 g of n-Docosyl Coumarate and allowed to stir for 30 minutes followed by the addition of 5 g of calcium stearate. The resulting solution was stirred for 1 hr at room temperature and dried.

### Example 4b

25 g of Isomaltulose was added to 100 mL purified water under stirring at room temperature. Slowly added 0.5 g of n-Octadecyl ferulate and allowed to stir for 30 minutes followed by the addition of 5 g of calcium stearate. The resulting solution was stirred for 1 hr at room temperature and evaporated to dryness.

### Example 4c

25 g of D-Allulose was added to 100 mL purified water under stirring at room temperature. Slowly added 1 g of n-trans-Caffeoyl-tyramine and allowed to stir for 30 minutes followed by the addition of 5 g of calcium stearate. The resulting solution was stirred for 1 hr at room temperature and freeze dried.

We claim:

1. A composition comprising Cinnamic acid derivatives with sucrose/sugar isomers having nutritive/therapeutic value, wherein, the Cinnamic acid derivatives is present in an amount of 2%-90% w/w and sucrose/sugar isomers present in an amount of 10%-90% w/w.

2. The composition as claimed in claim 1, wherein the said composition comprises nutraceutical/therapeutic excipients in an amount of 5-90%.

3. The composition as claimed in claim 1, wherein the Cinnamic acid derivatives are selected from Hydroxy Cinnamic acids, its esters and amides.

4. The composition as claimed in claim 3, wherein the Hydroxy Cinnamic acid derivatives (CA) are selected from Hydroxy Cinnamic acid, Ferulic acid, Caffeic acid, Sinapic acid, Chlorogenic Acid, Syringic acid p-Coumaric acid and the like.

5. The composition as claimed in claim 3, wherein the derivatives of Cinnamic acid esters are selected from alkyl/aralkyl alcohols esterified with Cinnamic acids having general formula CA-CO—OR wherein CA represents Cinnamic Acid and R represents C1-C22 alkyl chain, Ph-CH₂CH₂— or CA-CO—O(CH)nO-CO-CA; n=1-10 wherein the esters of Cinnamic acids includes branched alkyl groups such as Isopropyl, Iso-butyl cinnamate, Isopentyl and the like.

6. The composition as claimed in claim 3, wherein the derivatives of Cinnamic acid amides are selected from amides having general formula CA-CO—NHR wherein CA represents Cinnamic Acid and NHR represents Tyramine, Serotonin, Dopamine, Phenethylamine.

7. The compositions as claimed in claim 1, wherein the isomers of sugars are selected from monosaccharides or disaccharides.

8. The composition as claimed in claim 7, wherein the isomers of monosaccharides are selected from D Allose, D Allulose and D Tagatose.

9. The compositions as claimed in claim 7, wherein isomers of disaccharides are selected from Trehalulose, Turanose, Leucrose and Isomaltulose.

10. The composition as claimed in claim 2, wherein the composition may contain nutraceutical/therapeutic excipients selected from the group consisting of binders, diluents, lubricants, stabilizers, solubilizers, sustained release polymers, coloring agents, taste enhancers and the like.

11. The composition as claimed in any one of the claims 1 to 10, wherein the composition may be formulated into either oral solid, liquid or suspensions.

12. The composition comprising Cinnamic acid derivatives with sucrose isomers as claimed in any one of the preceding claims 1 to 11 as nutraceutical/therapeutic composition.

13. A process for preparation of the composition comprising;
i. Adding cinnamic acid derivatives in an amount of 2%-90% w/w to the aqueous solution of the sugar isomers in an amount of 10%-90% w/w;
ii. Adding suitable nutraceutical excipients in an amount of 5-90% w/w to the solution of step (i); and
iii. Agitating the solution of step (ii) to avoid heat-induced inversion of sucrose followed by drying to obtain the desired composition.

14. A method of enhancing the brain performance, reducing anxiety, improving the learning and focus without anxiety in a subject wherein the method comprises providing said subject with a composition comprising Cinnamic Acids or its derivatives with Sucrose Isomers as claimed in any one of the preceding claims in nutraceutically effective amount.

15. A method of treating inflammation in a subject wherein the method comprises providing said subject with a composition comprising Cinnamic Acids or its derivatives with Sucrose Isomers as claimed in any one of the preceding claims in therapeutically effective amounts, wherein the inflammation is selected from the inflammation related to the COX 2.

16. The composition comprising Cinnamic acid derivatives with sucrose isomers as claimed in any one of the preceding claims for enhancing the brain performance, reducing anxiety, improving the learning and focus without anxiety.

17. The composition comprising Cinnamic acid derivatives with sucrose isomers as claimed in any one of the preceding claims for treating inflammation, wherein the inflammation is selected from the inflammation related to the COX 2.

18. The composition comprising Cinnamic acid derivatives with sucrose isomers as claimed in any one of the preceding claims useful as nootropic performance enhancers and for the pain management.
